(19) United States
(12) Patent Application Publication
(10) Pub. No.: US 2025/0235191 A1
(43) Pub. Date: Jul. 24, 2025

(54) GRAPHICAL USER INTERFACE FOR BIOPSY DEVICE

(71) Applicant: DEVICOR MEDICAL PRODUCTS, INC., Cincinnati, OH (US)

(72) Inventors: Alex XU, Cincinnati, OH (US); George JIANG, Cincinnati, OH (US); Frank WU, Cincinnati, OH (US); Lirong QU, Cincinnati, OH (US); Jason LIU, Cincinnati, OH (US); Adair SONG, Cincinnati, OH (US); Christ CHEN, Cincinnati, OH (US)

(21) Appl. No.: 19/173,872

(22) Filed: Apr. 9, 2025

Related U.S. Application Data
(63) Continuation of application No. PCT/CN2022/124601, filed on Oct. 11, 2022.

(51) Int. Cl. A61B 10/02

(52) U.S. Cl. CPC A61B 10/0283; A61B 2560/0266; A61B 2560/045; A61B 2560/0487

(57) ABSTRACT

An apparatus includes a biopsy device, a valve assembly, and a control module. The valve assembly is configured to be in fluid communication with the biopsy device. The control module is configured to be in electrical communication with the biopsy device and in fluid communication with the valve assembly. The control module includes a display. The display is configured to provide instructions for: placing the biopsy device in electrical communication with the control module, inserting the valve assembly into the control module, and placing the valve assembly in fluid communication with the control module.

### PRIORITY STATEMENT

This application is a continuation of PCT/CN2022/124601 filed Oct. 11, 2022, now WO 2021/077477, published Apr. 18, 2024, the disclosure of which is incorporated by reference herein, in its entirety.

### BACKGROUND

Biopsy samples have been obtained in a variety of ways in various medical procedures using a variety of devices. Biopsy devices may be used under stereotactic guidance, ultrasound guidance, MRI guidance, PEM guidance, BSGI guidance, or otherwise. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device.

Merely exemplary biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 5,928,164, entitled “Apparatus for Automated Biopsy and Collection of Soft Tissue,” issued Jul. 27, 1999; U.S. Pat. No. 6,017,316, entitled “Vacuum Control System and Method for Automated Biopsy Device,” issued Jan. 25, 2000; U.S. Pat. No. 6,086,544, entitled “Control Apparatus for an Automated Surgical Biopsy Device,” issued Jul. 11, 2000; U.S. Pat. No. 6,162,187, entitled “Fluid Collection Apparatus for a Surgical Device,” issued Dec. 19, 2000; U.S. Pat. No. 6,432,065, entitled “Method for Using a Surgical Biopsy System with Remote Control for Selecting an Operational Mode,” issued Aug. 13, 2002; U.S. Pat. No. 6,626,849, entitled “MRI Compatible Surgical Biopsy Device,” issued Sep. 11, 2003; U.S. Pat. No. 6,752,768, entitled “Surgical Biopsy System with Remote Control for Selecting an Operational Mode,” issued Jun. 22, 2004; U.S. Pat. No. 7,442,171, entitled “Remote Thumbwheel for a Surgical Biopsy Device,” issued Oct. 8, 2008; U.S. Pat. No. 7,648,466, entitled “Manually Rotatable Piercer,” issued Jan. 19, 2010; U.S. Pat. No. 7,837,632, entitled “Biopsy Device Tissue Port Adjustment,” issued Nov. 23, 2010; U.S. Pat. No. 7,854,706, entitled “Clutch and Valving System for Tetherless Biopsy Device,” issued Dec. 1, 2010; U.S. Pat. No. 7,914,464, entitled “Surgical Biopsy System with Remote Control for Selecting an Operational Mode,” issued Mar. 29, 2011; U.S. Pat. No. 7,938,786, entitled “Vacuum Timing Algorithm for Biopsy Device,” issued May 10, 2011; U.S. Pat. No. 8,083,687, entitled “Tissue Biopsy Device with Rotatably Linked Thumbwheel and Tissue Sample Holder,” issued Dec. 21, 2011; and U.S. Pat. No. 8,118,755, entitled “Biopsy Sample Storage,” issued Feb. 21, 2012. The disclosure of each of the above-cited U.S. Patents is incorporated by reference herein.

Additional exemplary biopsy devices and biopsy system components are disclosed in U.S. Pub. No. 2006/0074345, entitled “Biopsy Apparatus and Method,” published Apr. 6, 2006; U.S. Pub. No. 2008/0146962, entitled “Biopsy System with Vacuum Control Module,” published Jun. 19, 2008; U.S. Pub. No. 2008/0214955, entitled “Presentation of Biopsy Sample by Biopsy Device,” published Sep. 4, 2008; U.S. Pub. No. 2008/0221480, entitled “Biopsy Sample Storage,” published Sep. 11, 2008; U.S. Pub. No. 2009/0131821, entitled “Graphical User Interface For Biopsy System Control Module,” published May 21, 2009; U.S. Pub. No. 2009/0131820, entitled “Icon-Based User Interface on Biopsy System Control Module,” published May 21, 2009; U.S. Pub. No. 2009/0216152, entitled “Needle Tip for Biopsy Device,” published Aug. 27, 2009; U.S. Pub. No. 2010/0113973, entitled “Biopsy Device with Rotatable Tissue Sample Holder,” published May 6, 2010; U.S. Pub. No. 2010/0152610, entitled “Hand Actuated Tetherless Biopsy Device with Pistol Grip,” published Jun. 17, 2010; U.S. Pub. No. 2010/0160819, entitled “Biopsy Device with Central Thumbwheel,” published Jun. 24, 2010; U.S. Pub. No. 2010/0160824, entitled “Biopsy Device with Discrete Tissue Chambers,” published Jun. 24, 2010; U.S. Pub. No. 2010/0317997, entitled “Tetherless Biopsy Device with Reusable Portion,” published Dec. 16, 2010; U.S. Pub. No. 2012/0109007, entitled “Handheld Biopsy Device with Needle Firing,” published May 3, 2012; U.S. Pub. No. 2012/0265095, entitled “Biopsy Device with Motorized Needle Firing,” published Oct. 18, 2012; U.S. Pub. No. 2012/0283563, entitled “Biopsy Device with Manifold Alignment Feature and Tissue Sensor,” published Nov. 8, 2012; U.S. Pub. No. 2012/0310110, entitled “Needle Assembly and Blade Assembly for Biopsy Device,” published Dec. 6, 2012; U.S. Pub. No. 2013/0041256, entitled “Access Chamber and Markers for Biopsy Device,” published Feb. 14, 2013; U.S. Pub. No. 2013/0053724, entitled “Biopsy Device Tissue Sample Holder with Bulk Chamber and Pathology Chamber,” published Feb. 28, 2013; U.S. Pub. No. 2013/0150751, entitled “Biopsy Device with Slide-In Probe,” published Jun. 13, 2013; U.S. Pub. No. 2013/0324882, entitled “Control for Biopsy Device,” published Dec. 5, 2013; U.S. Pub. No. 2013/0218047, entitled “Biopsy Device Valve Assembly,” published Aug. 22, 2013; and U.S. Pub. No. 2014/0039343, entitled “Biopsy System,” published Feb. 6, 2014. The disclosure of each of the above-cited U.S. Patent Application Publications is incorporated by reference herein.

In some biopsy devices, multiple components may be used to provide disposable and reusable elements of a given biopsy device. Additionally, some biopsy devices may be used with certain accessory components such as valves, associated tube sets, control cables, control modules, and/or etc. Thus, some biopsy devices may be associated with relatively complex setup procedures requiring multi-step assembly protocols. Thus, it may be desirable for such biopsy devices to be used with an automated set-up, wherein the biopsy device and associated components may be set-up for each patient quickly, easily, and with minimal opportunity for operator error. It may be desirable for a biopsy device to allow for fast connections and the possibility of confirmation of each connection. It may also be desirable to make apparent to a user when a connection has been lost.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an operator using a biopsy device, a control module, in combination with an ultrasonic imaging device for removing a tissue sample from a patient;

FIG. 2 depicts a perspective exploded view of the biopsy device of FIG. 1, the biopsy device being in communication with the control module depicted schematically;

FIG. 3 depicts a detailed perspective view of the control module of FIG. 1, the control module being configured to receive a valve assembly in a valve receptacle thereof;

FIG. 4 shows a perspective view of the valve assembly of FIG. 3;

FIG. 5 depicts a cross-sectional view of the valve receptacle of FIG. 3, the cross-section taken along line 5-5 of FIG. 3;

FIG. 6A depicts another cross-sectional view of the valve receptacle of FIG. 3, with the valve assembly being partially disposed within of the valve receptacle;

FIG. 6B depicts yet another cross-sectional view of the valve receptacle of FIG. 3, with the valve assembly being inserted into of the valve receptacle;

FIG. 6C depicts still another cross-sectional view of the valve receptacle of FIG. 3, with the valve assembly being fully seated within the valve receptacle;

FIG. 7 depicts a start-up visualization for display on the control module of FIG. 1:

FIGS. 8A and 8B depict a first visualization for display on the control module of FIG. 1;

FIGS. 9A and 9B depict a second visualization for display on the control module of FIG. 1;

FIGS. 10A and 10B depict a third visualization for display on the control module of FIG. 1;

FIGS. 11A and 11B depict a fourth visualization for display on the control module of FIG. 1:

FIG. 12 depicts a fifth visualization for display on the control module of FIG. 1;

FIG. 13 depicts an initialization visualization for display on control module of FIG. 1;

FIG. 14 depicts a sampling visualization for display on control module of FIG. 1;

FIG. 15 depicts a flow chart showing an exemplary process for using the visualizations of FIGS. 7-13.

### DETAILED DESCRIPTION

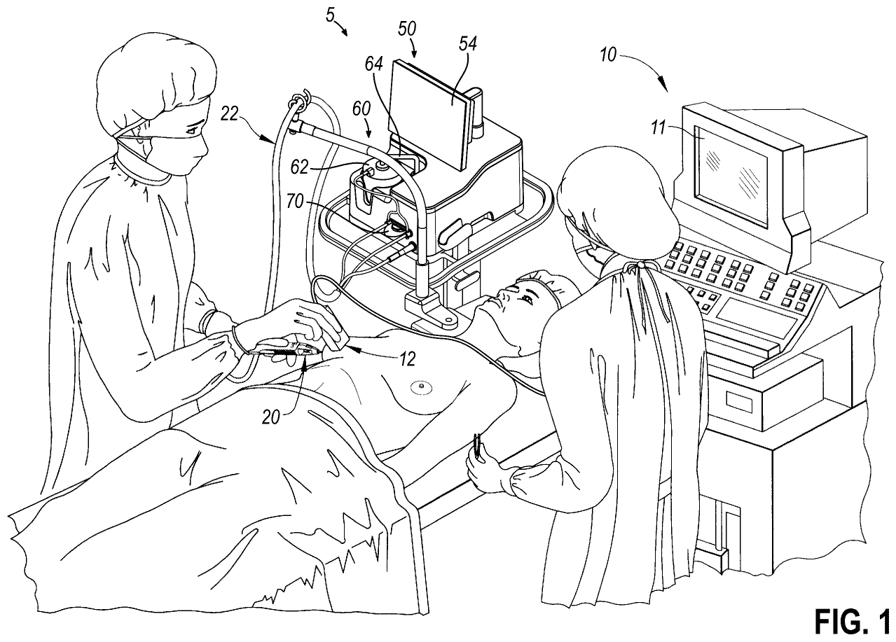

FIG. 1 shows an operator, an assistant, and a patient who is undergoing a breast biopsy. The operator is using a biopsy device (5) having a handpiece (20) (also referred to as an instrument) and a control module (50) (also referred to as a controller, a unit, and an assembly), which are operatively connected by a cord bundle (22). The operator may use biopsy device (5) in combination with an ultrasonic imaging device (10) having an imaging device handpiece (12) and an imaging device display (11). Imaging device (10) provides a real-time image of lesions, microcalcifications, and high-density masses within the breast tissue of the patient. The operator directs the imaging device handpiece (12) towards suspected tissue masses and guides the distal tip of handpiece (20) of biopsy device (5) adjacent to the suspected tissue for collection of one or more tissue samples. Control module (50) may include a visualization (54) for directing the operator in the sequence of operation of biopsy device (5). Visualization (54) may include a screen, a touchscreen, a projector, a light or any other indicator as reasonable foreseeable. In the present version, a fluid collection system (60) may be integrated into control module (50) for the removal of fluids from handpiece (20) or to otherwise provide operational control of handpiece (20). A valve assembly (70) is shown inserted into control module (50) and is provided for automatically regulating the communication of fluids between handpiece (20) and a vacuum canister (62). A vacuum pump line (64) connected to a vacuum source inside control module (50) supplies vacuum to vacuum canister (62).

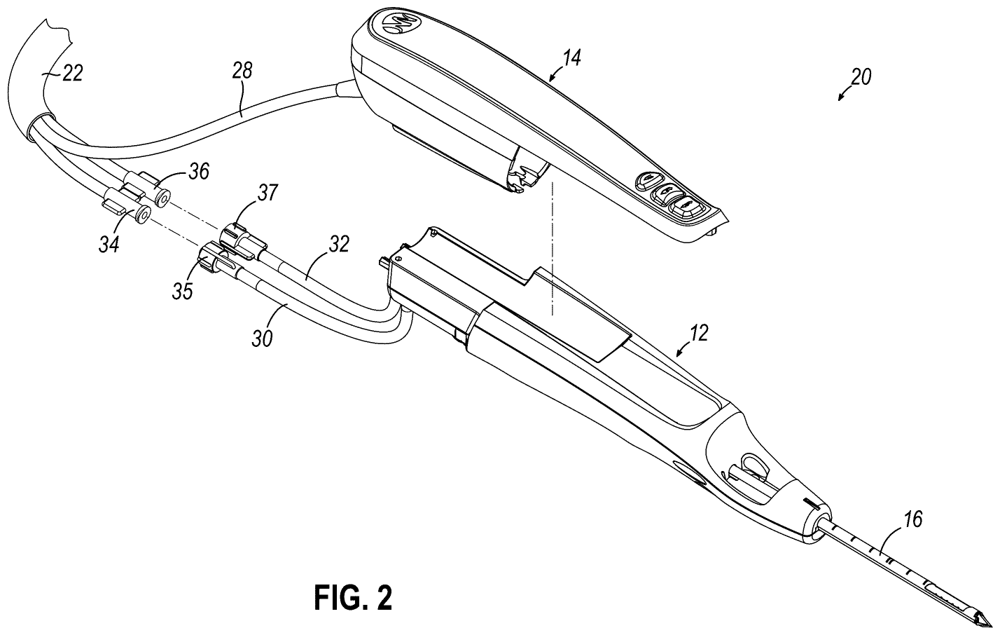

FIG. 2 shows bandpiece (20) in greater detail. As can be seen, cord bundle (22) may contain a control cord (28) for the electrical interface with control module (50), an optional first shaft and a second shaft (not shown) for operatively connecting handpiece (20) to a power transmission source (may be contained inside of control module (50)), and a first vacuum line (30) and a second vacuum line (32) for fluidly connecting handpiece (20) to fluid collection system (60). In some versions, first shaft and second shaft may be replaced with an electrical cable in electrical communication with a motor (not shown) in bandpiece (20). In such versions, transmission source may be omitted and instead one or more motors may be incorporated into holster (14), probe (12), or other portions of biopsy device (5). Although cord bundle (22) is shown and described herein as a combination of elements bundled together, it should be understood that in other versions, such elements may be unbundled with control cord (28), first vacuum line (30) and second vacuum line (32) being freely movable relative to each other. Alternatively, some elements may be bundled (e.g., first vacuum line (30) and second vacuum line (32)), while other elements may be un-bundled (e.g., control cord (28)). Cord bundle (22) may be bundled together by removable tie wraps or the like and can be easily detachable from handpiece fluid collection system (60), control module (50), and power transmission source (52) in order to clean and/or dispose of handpiece (20) or a portion of handpiece (20).

First vacuum line (30) and second vacuum line (32) may be optionally relatively short (several inches) compared to the overall length of cord bundle (22). Additionally, vacuum lines (30, 32) may be fluidly coupled to fluid collection system (60) by a first male connector (34), a first female connector (35), a second male connector (36), and a second female connector (37), respectively.

Handpiece (20) of the present example includes a probe (12) and a holster (14). A needle (16) extends distally from probe (12), and is generally configured for insertion into a patient's tissue to obtain one or more tissue samples. In some versions, the one or more tissue samples obtained by needle (16) may be deposited in a tissue sample holder (not shown) at the proximal end of probe (12). It should also be understood that the use of the term “holster” herein should not be read as requiring any portion of probe (12) to be inserted into any portion of holster (14). For instance, probe (12) may be removably secured to holster (14) by a variety of structures, components, features, etc. (e.g., bayonet mounts, latches, prongs, clamps, clips, snap fittings, etc.). Furthermore, in some versions of handpiece (20), probe (12) and holster (14) may be of unitary or integral construction, such that the two components cannot be separated. By way of example only, in versions where probe (12) and holster (14) are provided as separable components, probe (12) may be provided as a disposable component, while holster (14) may be provided as a reusable component. Still other suitable structural and functional relationships between probe (12) and holster (14) will be apparent to those of ordinary skill in the art in view of the teachings herein.

Probe (12) and/or holster (14) may include suitable electronics to detect when holster (14) and probe (12) have been coupled and to communicate whether probe (12) and holster (14) have been coupled or not to control module (50). As will be described in greater detail below, detection of probe (12) and holster (14) coupling may be desirable to aid a user during certain setup procedures. In addition, or in the alternative, such detection may be desirable to identify error conditions during a biopsy procedure such as a decoupling between probe (12) and holster (14). By way of example only, suitable electronics may include sensors disposed in either probe (12) or holster (14). Such sensors may include, for example, RFID tag/sensor combinations, proximity sensors, light sensors, hall effect sensors, and/or etc. Alternatively, in some versions, probe (12) and holster (14) coupling may be detected remotely using control module (50) by the detection of electrical continuity between probe (12) and holster (14). Of course, other suitable electronics suitable to detect probe (12) and holster (14) coupling will be apparent to those of ordinary skill in the art in view of the teachings herein.

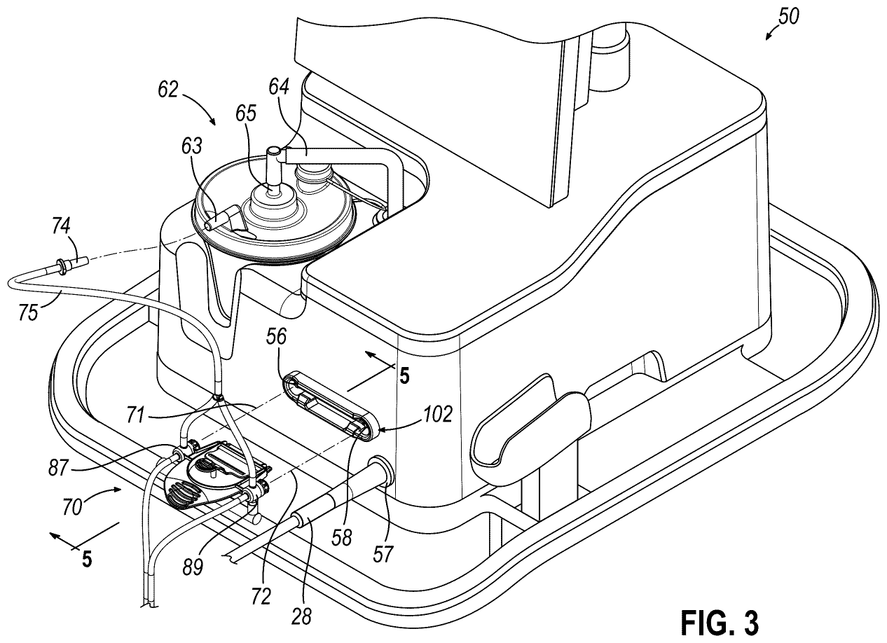

FIG. 3 shows control module (50) in greater detail. As can be seen, control module (50) of the present version includes a valve receptible (102). Valve receptacle (102) defines an opening within a portion of control module (50) that is generally configured to receive a portion of valve assembly (70). As will be described in greater detail below, the interior of valve receptacle (102) may include one or more actuators (56, 58) configured to engage and to actuate valve assembly (70) using one or more motors disposed within control module (50). Thus, it should be understood that valve receptacle (102) may define a shape that is generally complementary to the shape of valve assembly (70) such that valve assembly (70) may be received therein.

As will be described in greater detail below, valve assembly (70) may be aligned along a first longitudinal axis (71) and a second longitudinal axis (72) for insertion into valve receptacle (102) contained within control module (50). A common distal line (75) having a stem connector (74) fluidly connects valve assembly (70) to a second canister port (63) of vacuum canister (62). Vacuum pump line (64) is fluidly connected to a first canister port (65) of vacuum canister (62). A control cord receptacle (57) is provided for detachably connecting control cord (28) to control module (50) (see FIG. 2).

Still referring to FIG. 3, valve assembly (70) includes a first valve (87) that defines first longitudinal axis (71), and a second valve (89) that defines second longitudinal axis (72), which is parallel to first longitudinal axis (71). In some versions, valve receptacle (102) is configured such that valve assembly (70) may only be received therein when first longitudinal axis (71) and second longitudinal axis (72) are aligned with valve receptacle (102). As will be described in greater detail below, such a configuration may be desirable to promote proper seating of valve assembly (70) within valve receptacle (102). First valve (87) and second valve (89) are rotary valves in the present version, although other types of valves may be used in addition to, or in lieu of, rotary valves. First valve (87) may also be referred to as a first rotary valve, and second valve (89) may also be referred to as a second rotary valve.

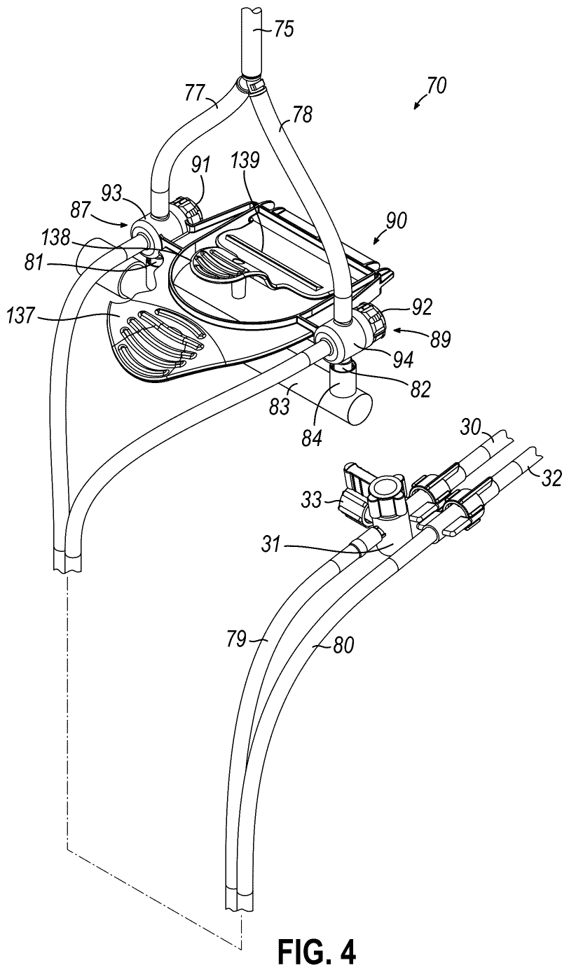

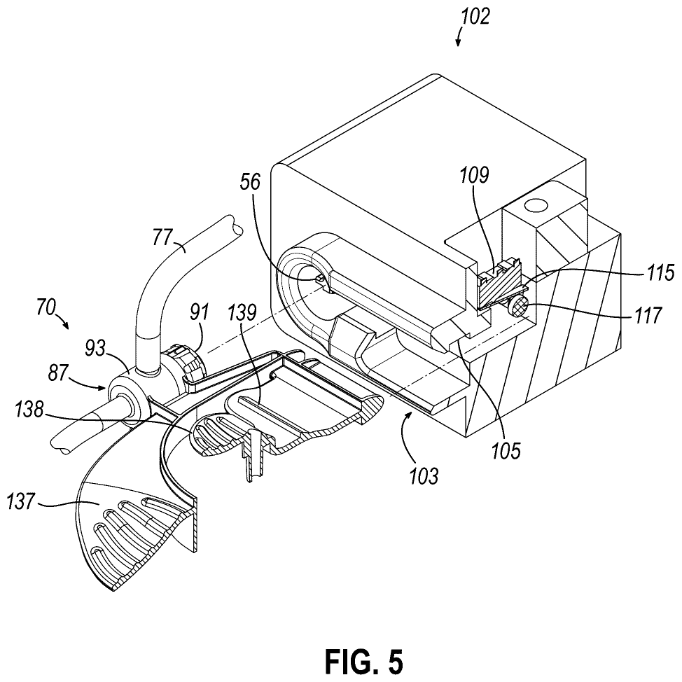

FIG. 4 shows valve assembly (70) in greater detail. As can be seen, first valve (87) and second valve (89) may be affixed in a generally parallel alignment onto a valve frame (90) having a valve frame handle (137). Valve assembly (70) further includes a vent assembly (not shown) (also referred to as an air vent) affixed to a latch lever (138) cantileverly attached to frame (90) and disposed between first valve (87) and second valve (89). Valve assembly (70) includes a lip (139) which is cantilevered on frame (90) along with latch lever (138). Lip (139) may be configured to engage a corresponding lip cavity (105) (see FIG. 5) disposed within a portion of valve receptacle (102), as will be described in greater detail below. First valve (87) may include a first valve spool (91) rotatably inserted into a first valve housing (93). Second valve (89) may include a second valve spool (92) rotatably inserted into a second valve housing (94). Valve assembly (70) further includes a first distal line (77) fluidly connected to first valve (87), and a second distal line (78) fluidly connected to second valve (89). First distal line (77) may join second distal line (78), which may be fluidly connected to common distal line (75) shown in FIG. 3. Valve assembly (70) may further include a first vent line (81) fluidly connected to first valve (87), and a second vent line (82) fluidly connected to second valve (89). First vent line (81) and second vent line (82) may be joined at a T-connector (84), which may be fluidly connected by a vent tube (83) to vent assembly.

Valve frame (90), first valve (87), second valve (89), latch lever (138), and valve handle (137) may be made from a medical grade, rigid, injection molded plastic such as polycarbonate. All of the fluid carrying lines described for valve assembly (70) (including first distal line (77), second distal line (78), first fluid line (79), second fluid line 80, first vent line (81), second vent line (82), vent tube (83)) may be made of an economical, flexible, medical grade material such as polyvinyl chloride (PVC). In some versions, it may be desirable to make first valve spool (91) and second valve spool (92) of a rigid, medical grade plastic such as polyethylene to maintain the integrity of first and second valve spool (91, 92) during operation.

First vacuum line (30) of handpiece (20) shown in FIG. 2 may be fluidly connected to first valve (87) via a first fluid line (79), shown in FIG. 3. As shown in FIG. 4, first female connector (35) may include an injection port (33) (not shown in FIG. 2), which may be fluidly connectable to first vacuum line (30) by actuation of a 3-way valve (31). Injection port (33) provides the operator a convenient place to inject solutions such as lidocaine for local anesthesia into the tissue of the patient. Second vacuum line (32) of biopsy device (5) shown in FIG. 2 may be fluidly connected to second valve (89) via a second fluid line (80) shown in FIG. 4.

In the present version of valve assembly (70), first valve (87) and second valve (89) may be configured as rotational type valves. This type of valve may be desirable in some circumstances over other types of valves for a variety of reasons. For instance, in some circumstances, a rotational type valve may provide ease of setting up the fluid collection system by the operator. Additionally, rotational type valves may be desirable to reduce costs of valve assembly (70) by permitting the use of lower cost tubing or other structures. In some versions, one or more portions of valve assembly (70) may be configured in accordance with the teachings of U.S. Pat. Nos. 6,162,187; and 9,724,076, the disclosures of which are incorporated by reference herein.

FIG. 5 shows the interior of valve receptacle (102) in greater detail. As can be seen, valve receptacle (102) may include a valve port (103), which may be configured to receive valve assembly (70). Inside valve port (103), a portion of valve port (103) can define lip cavity (105). Lip cavity (105) may be configured to engage lip (139) of valve assembly (70) to secure valve assembly (70) to valve receptacle (102) such that first valve (87) and second valve (89) are able to be controlled by control module (50). Lip cavity (105) of the present version is defined by an upper portion of valve port (103) and extends transversely relative to a receiving axis defined by valve port (103). In other versions, lip cavity (105) may be located anywhere in valve port (103) for valve assembly (70) to couple with valve receptacle (102). In addition, or in the alternative, in some versions lip cavity (105) may include a plurality of cavities or other engagement features rather than a single feature as shown. In such versions, lip (139) of valve assembly (70) may likewise be divided into a plurality of lips or other engagement features corresponding to each lip cavity (105).

Valve port (103) may also include at least one power actuator, such as first actuator (56) and second actuator (58) (shown in FIG. 3), which may be configured to engage with a valve, such as first valve (87), to throttle material coming from or going to handpiece (20). Such actuators (56, 58) may extend axially within valve port (103) to engage each valve (87, 89) of valve assembly (70). As will be understood, actuators (56, 58) may be rotatable within valve port (103) to control the pneumatic state of each respective valve (87, 89). Thus, it should be understood that each actuator (56, 58) may include keys, indentations, or other features to transmit rotation from each actuator (56, 58) to each respective valve (87, 89). Although not shown, it should be understood that control module (50) may include one or more motors to control rotation of actuators (56, 58).

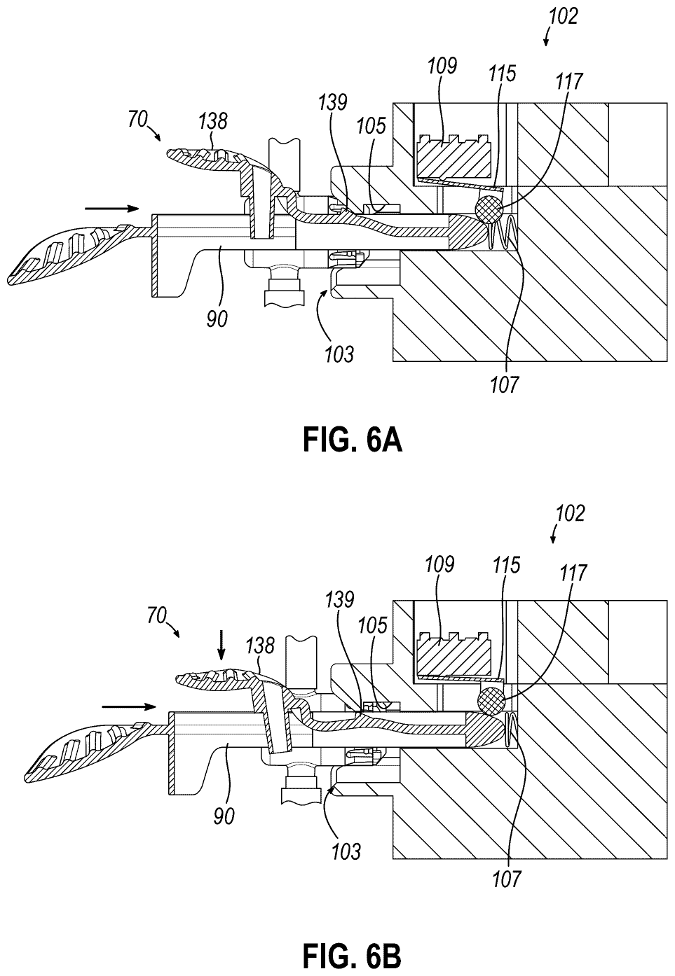

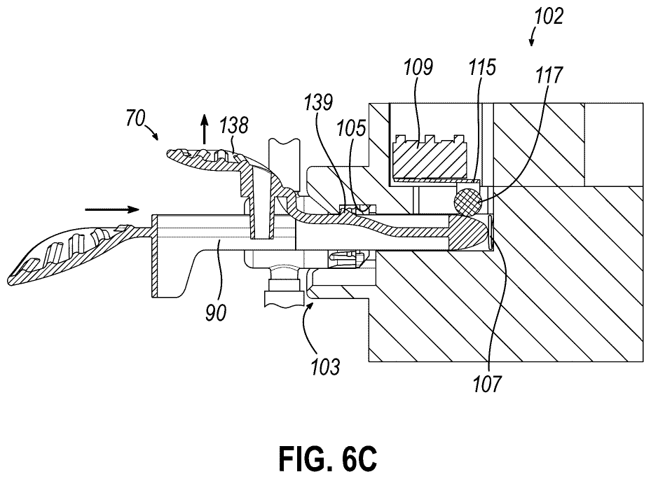

In some versions, it may be desirable to include certain ejection features within the interior of valve port (103). For instance, at least one valve port spring (107) (see FIGS. 6A, 6B, and 6C) may be located in valve port (103). Valve port spring (107) may be configured as a coil spring extending axially within valve port (103) from an interior wall opposite the opening defined by valve port (103). Such a valve port spring (107) may be configured to apply an outward force on valve assembly (70) as described below.

Valve port (103) can further include a valve switch (109). Valve switch (109) may be configured to communicate with control module (50) whether valve assembly (70) has been properly inserted into valve receptacle (102). In particular, valve switch (109) may be configured to engage with valve assembly (70) once valve assembly (70) is inserted into valve receptacle (102) and once lip (139) enters lip cavity (105).

In the present version, valve switch (109) can be a momentary normally open switch which transitions to an electrically closed position once valve assembly (70) is inserted into valve receptacle (102). Valve switch (109) may include an arm (115), which includes a roller (117) configured to contact valve assembly (70). When roller (117) contacts valve assembly (70), arm (115) rotates towards a closed position which then triggers valve switch (109) to transition from open position to closed position. Once in the closed position, valve switch (109) communicates to control module (50) that the valve has been inserted and control module (50) will then be operable to control first actuator (56) and second actuator (58). Once valve assembly (70) is removed from valve receptacle (102), valve switch (109) transitions from closed position back to open position and control of valve assembly (70) ceases. When valve port spring (107) is exerting an outward force on valve assembly (70), movement on valve assembly (70) relative to valve receptacle (102) is limited and the outward force also aids in removal of valve assembly (70) from valve receptacle (102) by pushing valve assembly (70) out of valve receptacle (102) when lip (139) disengages with lip cavity (105), as shown below.

Although valve switch (109) is shown in the present version as a spring-loaded switch including arm (115) and roller (117), it should be understood that in other versions, various alternative switches and switch configurations may be used in addition to, or in lieu of, valve switch (109). For instance, such alternative switches may include pressure sensitive switches, barrel or linear switches, and/or etc. In still other versions, other suitable position detecting mechanisms may be used instead of a switch-based configuration. By way of example only, suitable sensors may include light or image based sensors, magnetic sensors, RFID sensors, proximity sensors, pressure-based sensors, and/or etc.

FIGS. 6A through 6C show an example of a use of valve assembly (70) in connection with valve receptacle (102) and valve switch (109). FIG. 6A shows the beginning of valve assembly (70) being inserted into the valve receptacle (102). In this position, valve assembly (70) may be oriented in an upright position with latch lever (138) superior to the remaining portion of valve assembly (70). Specifically, latch lever (138) being superior to frame (90) aids in operator access to latch lever (138).

FIG. 6B shows valve assembly (70) partially inserted into valve receptacle (102) such that latch lever (138) and lip (139) are deflected towards the remaining portion of valve assembly (70). Deflecting latch lever (138) and lip (139) may be accomplished by either the operator pressing on latch lever (138) manually, which deflects latch lever (138) and lip (139) downwards or, with an adequate amount of lateral force on valve assembly (70) towards valve receptacle (102), the outer surface of valve receptacle (102) will press against lip (139) and thus force lip (139) and latch lever (138) to deflect towards frame (90) and allow lip (139) to translate towards lip cavity (105). Once valve assembly (70) has been properly inserted into valve receptacle (102), valve switch (109) transitions into closed position which signals to control module (50) that valve assembly is engaged and then control module is capable of making first and second actuators (56, 58) operational.

FIG. 6C shows valve assembly (70) fully inserted into the valve receptacle (102). Once fully inserted, lip (139) enters into lip cavity (105) such that valve assembly (70) is restricted from being removed from valve receptacle (102) until either latch lever (138) is pressed and lip (139) is deflected out of lip cavity (105) or until enough outward force is applied to valve assembly (70) such that valve assembly (70) is forced out of valve receptacle (102). Once valve assembly (70) is no longer engaged in valve receptacle (102), valve switch (109) can transition from closed position to open position to communicate to control module (50) that valve assembly (70) is no longer engaged and then control module (50) is capable of making first and second actuators (56, 58) non-operational.

In some versions, it may be desirable to communicate to an operator certain procedural steps for setting up biopsy device (5), and to confirm that one or more steps for setting up biopsy device (5) has been performed. As described above, biopsy device (5) may be used in combination with a variety of interconnected features such as probe (12), holster (14), chord bundle (22), valve assembly (70), control module (50), and/or etc. Thus, setup of biopsy device (5) may include a variety of connections that may be performed in one or more particular orders. Accordingly, communication of such operations for setup and confirming completion of each operation may allow an operator to begin using the biopsy device (5) without any complicating factors or delays. In some versions, such communications may be completed through the use of visualization (54). The following includes examples of how visualization (54) can communicate instructions for connecting and for confirmation that adequate connections have been completed. Although certain specific features are described herein, it should be understood that such features may be combined with other features described herein without departing from the spirit of the versions described herein.

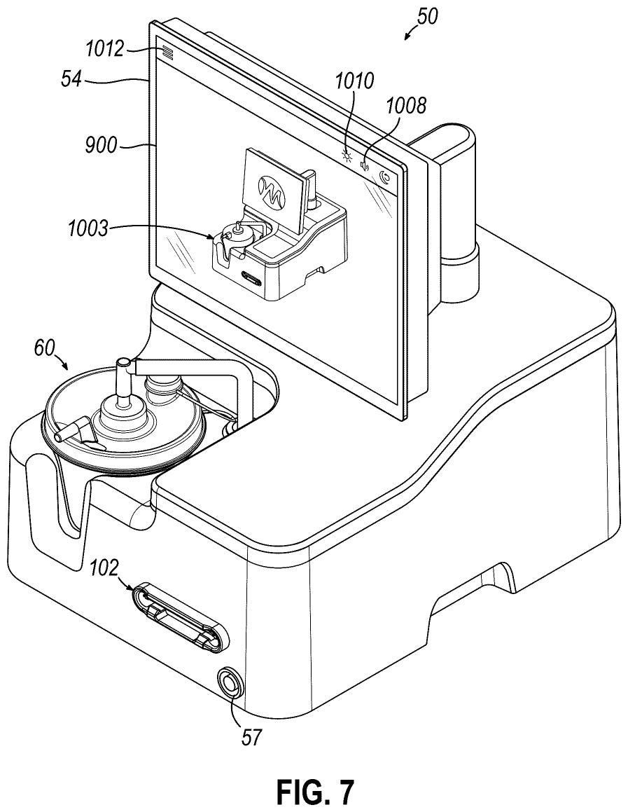

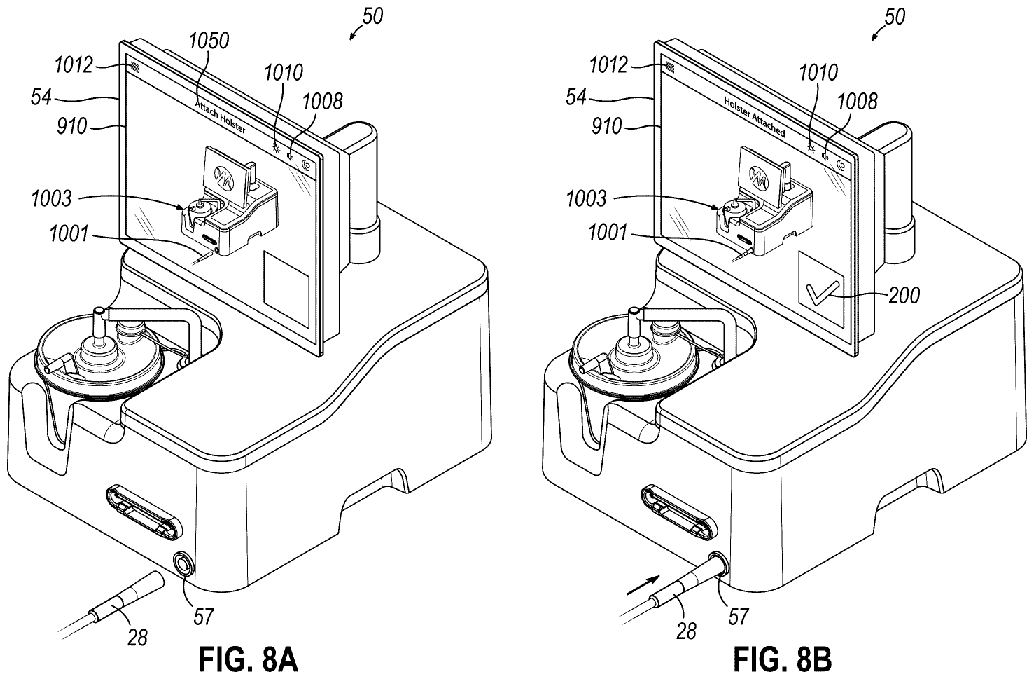

FIGS. 7-14 show exemplary elements (900, 910, 920, 930, 940, 950, 960, 970) that may be displayed on visualization (54) during set-up, initialization, and use of biopsy device (5). Visualization (54) may be capable of showing any aspect of the setup of biopsy device (5) including all or portions of elements (900, 910, 920, 930, 940, 950, 960, 970) either individually or in combination with each other. Visualization (54) may be capable of aiding in the setup of biopsy device (5) in any order and may be capable of adjusting in real-time the setup order and current step based on surrounding circumstances and one or more sensors included in biopsy device (5). Visualization (54) may be capable of showing text, symbols, and static or dynamic graphical representations of components of biopsy device (5) such that operator may be instructed to any remaining setup step or error during setup or use of biopsy device (5). Visualization (54) may be lit to be seen in low light situations or from distances by operator or may be projected onto a nearby wall. Visualization (54) may also illuminate or be color matched to a portion of biopsy device (5) to allow for proper connections of biopsy device (5). In particular, element (900) shown in FIG. 7 includes a graphical representation (1003) of control module (50), a confirmation that control module (50) is powered and that the set-up procedure can begin. Element (900) may provide an operator with an option to input the appropriate procedure or one or more peripheral devices that the operator would like visualization (54) to show a set-up sequence for. Visualization (54) can then guide the operator on the appropriate set-up steps.

Any one or more of elements (900, 910, 920, 930, 940, 950, 960, 970) may also include a volume adjustment button (1008), a brightness adjustment button (1010), and/or a settings adjustment button (1012). It should be understood that, while the term “button” is used herein, the same should not be read as requiring an electromechanical button that has a movable feature. The term may include interactive icons and other features of a flat touchscreen, etc. If the operator taps volume adjustment button (1008), a sub-element can pop up enabling the operator to select a volume level for audio feedback emitted by vacuum control module (not shown). If the operator taps brightness adjustment button (1010), a sub-element can pop up enabling the operator to select a brightness level for visualization (54). If the operator taps settings adjustment button (1012), a sub-element can pop up enabling the operator to adjust various settings for control module (50) (e.g., language, etc.).

Once the set-up procedure begins, it may first be desirable to connect control cord (28) of biopsy device (50) to control module (50). Performing this step first in some versions may be desirable to provide power to biopsy device (5), which may be used to power sensors, switches, and other components used for performing other set-up procedure steps. Element (910) shown in FIGS. 5A and 5B is generally configured to guide an operator through connection of control cord (28) to control module (50). In particular, element (910) includes a graphical representation (1003) of control module (50) with control cord receptacle (57) and a graphical representation (1001) of control cord (28), with a textual instruction (1050) to attach control cord (28) of holster (14) to control cord receptacle (57). Element (910) may include either an animation of graphical representations (1001, 1003) of control cord (28) being inserted into control cord receptacle (57), two separate images; one with control cord (28) not inserted into control cord receptacle (57) and one with control cord (28) inserted into control cord receptacle (57), or may include either image by itself.

Once the operator attaches control cord (28) to control cord receptacle (57), visualization (54) can display an indication (200) (also referred to as an indicator or feedback element) to confirm that holster (14) has been detected and that control module (50) is in communication with holster (14). Indications (200, 205, 210, 215) may be operable to instruct operator that a portion of a step has been completed. Indications (200, 205, 210, 215) may be embodied as a check mark, a green background, a circle, or any other mark or symbol reasonably foreseeable to indicate completion of a portion of a step to operator. Indications may also be presented as or in combination with visual indications as auditory or tactile indications. It should therefore be understood that control module (50) includes circuitry, as described above, configured to sense when holster (14) is attached to control module (50). Although indication (200) is shown in the present example as visual indicator confirming the completion of control cord (28) attachment, it should be understood that alternative forms of confirmation may be used in some versions in addition to, or in lieu of indication (200). For instance, in some versions confirmation may be facilitated by audible tones, tactile feedback, illumination of one or more accessory lights, and/or etc.

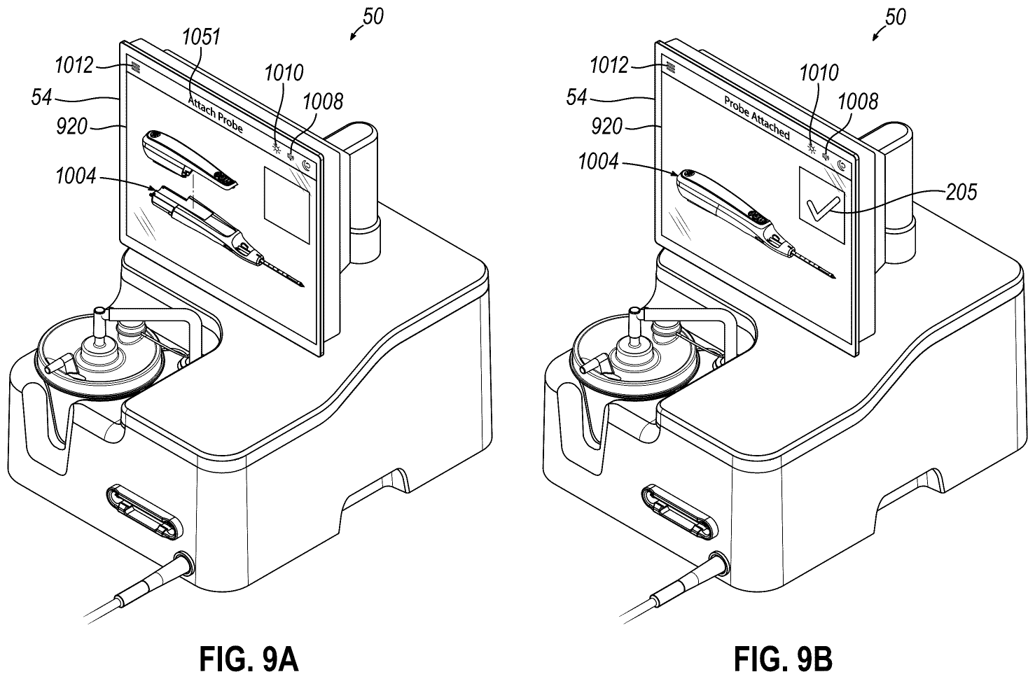

After attachment of control cord (28) to control cord receptacle (57), visualization (54) then transitions to clement (920) shown in FIGS. 9A and 9B. The transition of visualization (54) from element (900) to element (920) may occur automatically either upon attachment of control cord (28) or after a predetermined delay (e.g., a delay of 2 to 30 seconds). Alternatively, in some versions, visualization (54) may present an operator input button used to initiate the transition so that an operator may confirm that the operator is ready for the next procedural step.

Element (920) includes a graphical representation (1002) of holster (14) and a graphical representation (1004) of probe (12), with a textual instruction (1051) to attach probe (12) to holster (14). Element (920) may include either an animation of graphical representations (1002, 1004) of probe (12) being inserted into holster (14), two separate images; one with probe (12) not inserted into holster (14) and one with probe (12) inserted into holster (14), or may include either image by itself. It should therefore be understood that control module (50), probe (12) and/or holster (14) may include circuitry configured to sense when probe (12) is attached to holster (14). For instance, in some examples probe (12) may contain a magnet and holster (14) may contain a sensor configured to detect the magnetic field generated by the magnet in probe (100). In still other versions, control module (50) may be configured to detect certain changes in voltage and or current associated with probe (12)/holster (14) coupling. Various other forms that such circuitry may be apparent to those of ordinary skill in the art in view of the teachings herein. Optionally, element (920) may include volume adjustment button (1008), brightness adjustment button (1010), and settings adjustment button (1012), as described above.

Once an operator attaches probe (12) to holster (14), visualization (54) displays indication (205) (also referred to as an indicator or feedback element) to confirm that the attachment has been detected and that control module (50) is in communication with probe (12). As with the confirmation referred to above with respect to control cord (28) attachment, confirmation of probe (12) coupling may likewise be confirmed using a variety of specific indications (205) such as graphical indicators, audible indicators, tactile indicators, and/or etc. As similarly discussed above, such confirmation indicators may be used alone or in combination with each other.

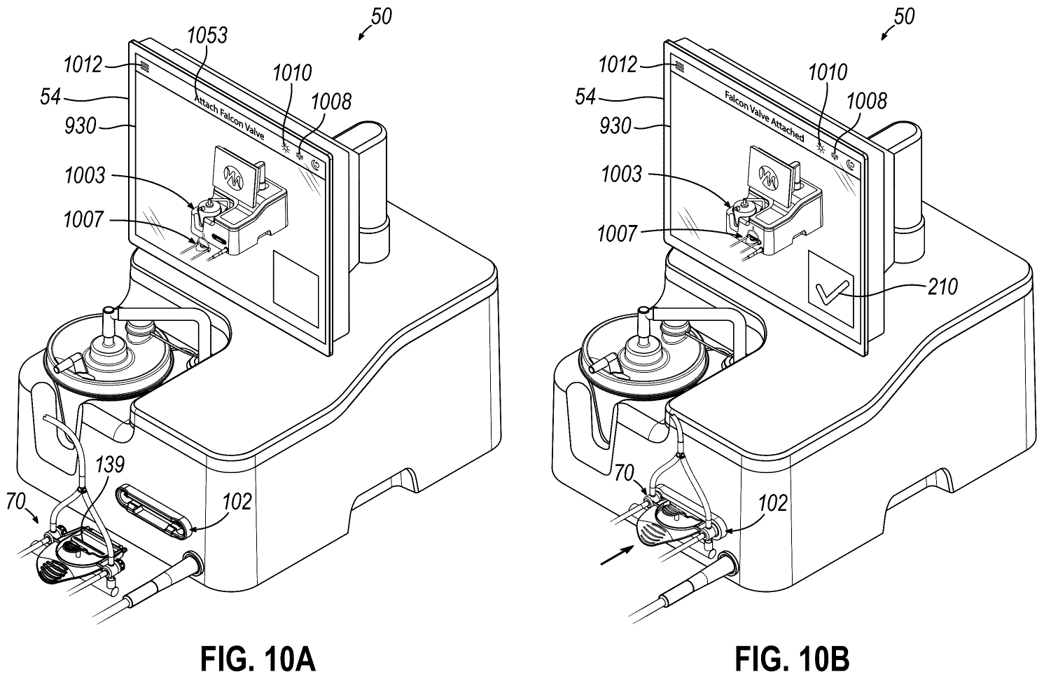

Once an operator attaches probe (12) to holster (14), visualization (54) may transition to element (930) shown in FIGS. 10A and 10B. The transition of visualization (54) from element (920) to element (930) may occur automatically either upon attachment of probe (12) to holster (14) or after a predetermined delay (e.g., a delay of 2 to 30 seconds). Alternatively, in some versions, visualization (54) may present an operator input button used to initiate the transition so that an operator may confirm that the operator is ready for the next procedural step.

Element (930) shown in FIGS. 10A and 10B includes a graphical representation (1007) of valve assembly (70) and a graphical representation (1003) of control module (50) with valve receptacle (102), with a textual instruction (1053) to attach valve assembly (70) to valve receptacle (102). Element (930) may include either an animation of graphical representations (1003, 1007) of valve assembly (70) being inserted into control module (50), two separate images; one with valve assembly (70) not inserted into control module (50) and one with valve assembly (70) inserted into control module (50), or may include either image by itself. The animation may include depictions of graphical representations (1003, 1007) of valve assembly being aligned with valve receptacle (102) and an operator inserting lip (139) into lip cavity (105).

Once an operator attaches valve assembly (70) to valve receptacle (102), visualization (54) displays an indication (210) (also referred to as an indicator or feedback element) to confirm that the attachment has been detected and that control module (50) is in communication with valve assembly (70). As described above, control module (50) may be configured to sense when valve assembly (70) has been inserted into valve receptacle (102). Valve receptacle (102) may be capable of detecting valve assembly (70) using valve switch (109) or another sensor capable of detecting valve assembly (70), such as; a proximity switch, a light sensor, a pressure switch, a hall effect sensor, a magnetic sensor, or any other sensor reasonable foreseeable in the art. Valve receptacle (102) may also be capable of detecting when valve assembly (70) has been partially inserted or when it has become uncoupled. Element (930) may then instruct operator to insert valve assembly (70) further into valve receptacle (102). Valve switch (109) may be normally open or normally closed and may be either a momentary switch or a maintained switch.

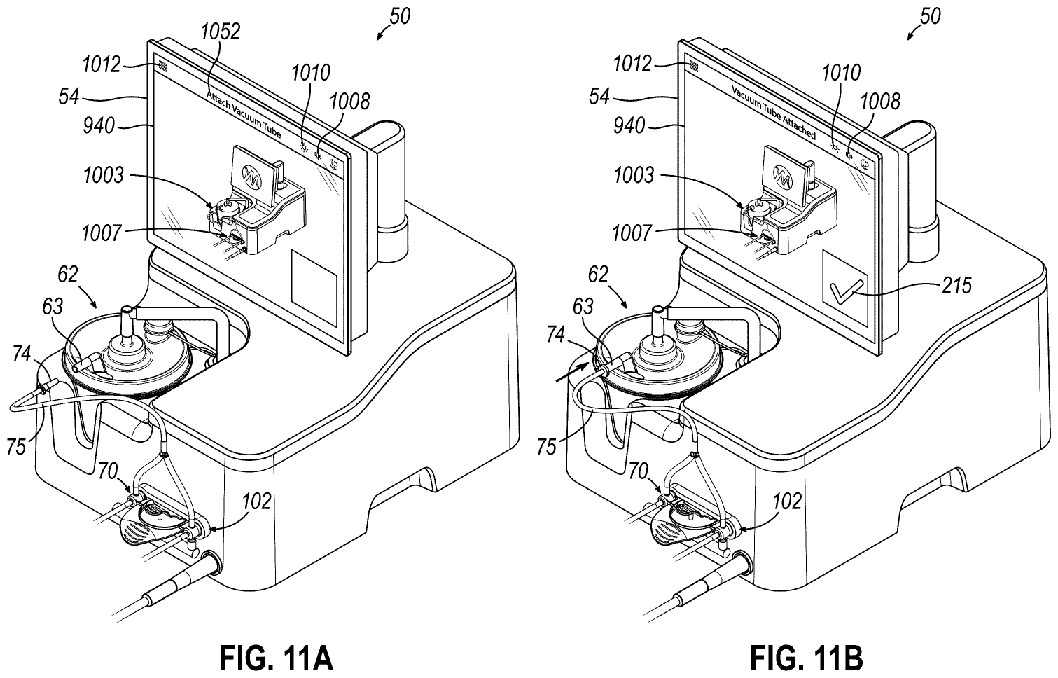

Visualization (54) then can transition to element (940) shown in FIGS. 11A and 11B. The transition of visualization (54) from element (930) to element (940) may occur automatically either upon attachment of valve assembly (70) or after a predetermined delay (e.g., a delay of 2 to 30 seconds). Alternatively, in some versions, visualization (54) may present an operator input button used to initiate the transition so that an operator may confirm that the operator is ready for the next procedural step.

Element (940) shown in FIGS. 11A and 11B includes a graphical representation (1003) of control module (50) with vacuum canister (62) with second canister port (63) and a graphical representation (1007) of valve assembly (70) with stem connector (74) from distal line (75), with a textual instruction (1052) to attach valve assembly (70) to vacuum canister (62). Element (940) may include an animation of graphical representations (1003, 1007) of valve assembly (70) being inserted into control module (50). In this version, animation may begin with stem connector (74) of valve assembly (70) being disconnected from second canister port (63) of control module (50) and then gradually show stem connector (74) being coupled with second canister port (63). In an alternative version, element (940) may show two separate images; one with stem connector (74) not coupled with second canister port (63) and one stem connector (74) coupled with second canister port (63), thus illustrating the need to transition from one scenario to another. Element (940) may also include either image by itself.

Once an operator attaches valve assembly (70) to vacuum canister (62), visualization (54) displays an indication (215) (also referred to as an indicator or feedback element) to confirm that the attachment has been detected and that vacuum canister (62) is in fluid communication with valve assembly (70). Thus, control module (50) may be configured to detect when vacuum canister (62) is in fluid communication with valve assembly (70). Such detection may be either by electrical communication, such as a switch, or by monitor the fluid resistance against the vacuum canister. Control module (50) may be capable of detecting a large pressure drop, such as when valve assembly (70) is disconnected from vacuum canister (62) and thus inform operator that attachment is needed to maintain vacuum at probe (12). Visualization (54) then transitions away from the set-up elements and into an operation mode for conducting the intended procedure.

The set-up steps may be performed in any order and are not restricted to the order depicted above. For instance, valve assembly (70) may be attached to vacuum canister (62) prior to valve assembly (70) being attached to valve receptacle (102). In another instance, probe (12) may be attached to holster (14) prior to holster (14) being attached to control module (50).

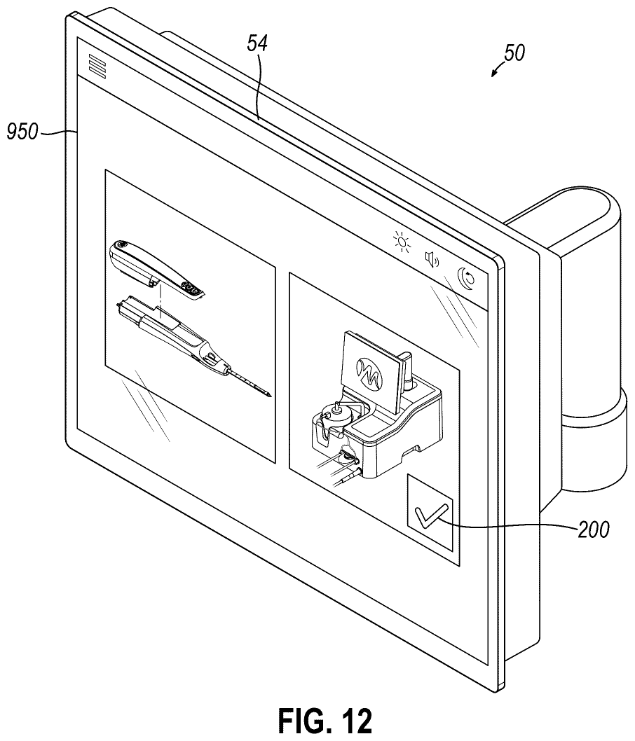

In some versions, it may be desirable to show a combination of elements (910, 920, 930, 940) described above on a single visualization (54) or screen. Such combination visualization (54) may be desirable to promote efficiency of operation by showing multiple procedure steps at a single time. This configuration may be particularly suitable for certain procedural steps that may be performed together or contemporaneously. For instance, element (950) shown in FIG. 12 includes graphical representations of multiple set-up steps being shown on the same element. Separate from the prior set-up elements which illustrated individual set-up steps, visualization (54) may also show multiple steps at the same time. For instance, element (950) depicts attachment of probe (12) to holster (14), attachment of valve assembly (70) with valve receptacle (102), and attachment of valve assembly (70) to vacuum canister (62) all on visualization (54) at the same time. Of course, other combinations of set-up steps may be shown on visualization (54) at the same time. Confirmation that a set-up step has been performed is shown with separate indications (200, 205, 210, 215) near the area on the visualization (54) which represented that step. Visualization (54) may maintain the configuration shown in FIG. 12 (or other suitable combinations) until all set-up steps illustrated on element (950) have been properly performed before advancing.

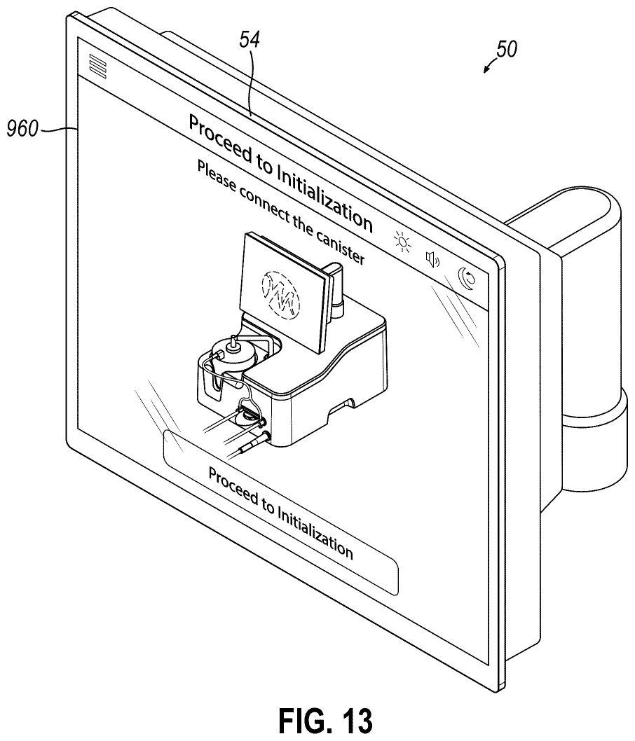

FIG. 13 shows initialization element (960) which may be shown once all set-up steps have been properly completed and control module (50) is in complete communication with handpiece (20). Before entering into operation mode, control module (50) may conduct an initialization step which may be indicated to operator on visualization (54) or may be done as a background function without operator's knowledge. When at the initialization step, control module (50) may conduct a series of checks where each function of biopsy device (5) is driven from one extreme limit to another extreme limit. Driving each function across the range of capabilities may allow control unit (50) to detect a malfunction or an improper connection before the operation begins and may allow control unit (50) to set a beginning home position for each function. The initialization of each function may also be performed upon the completion of each set-up step. For instance, once valve assembly (70) has been coupled to valve receptacle (102), control module (50) may perform an initialization step on each valve (87, 89) of valve assembly (70) by driving each valve across a range from completely closed to completely open and then may set each valve (87, 89) to a starting position which may be completely closed, completely open, or at an intermediate position.

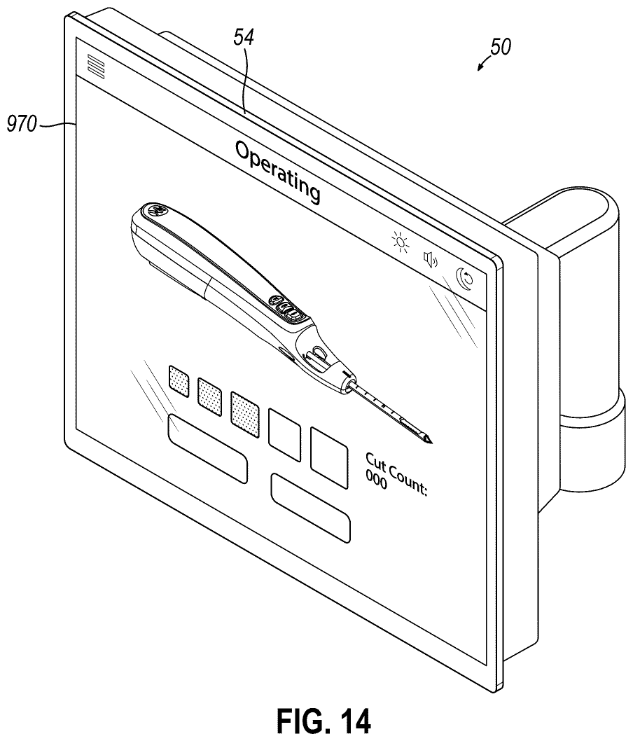

FIG. 14 shows sampling element (970) which may be shown once all set-up steps have been properly completed and may be shown once all initialization steps have been performed and once control module (50) is in complete communication with handpiece (20). Should also portion of handpiece (20) come uncoupled from control module (50) during operation, visualization (54) can warn the operator that handpiece has at least partially become uncoupled and has lost communication. Visualization (54) may then return to display the appropriate set-up or initialization element (900, 910, 920, 930, 940, 950, 960) which is needed to put control module (50) back into complete communication with handpiece (20). Once the appropriate portion of handpiece (20) has regained communication with control module (50), control module (50) may cycle through the remaining communications with handpiece (20) to confirm communication before returning to operation element (970).

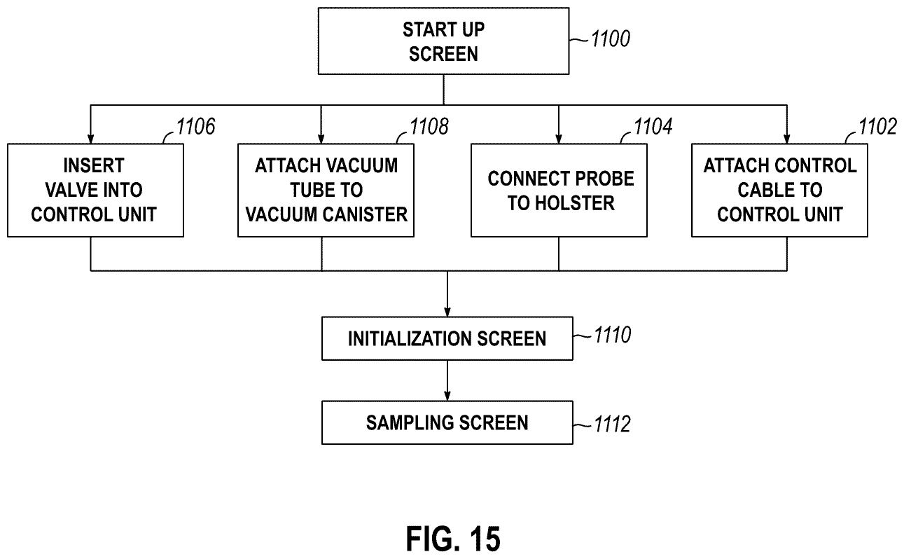

FIG. 15 shows a flow chart of an example set-up procedure using elements (900, 910, 920, 930, 940, 950, 960, 970) described above for core sampling biopsy device (5). As can be seen, block (1100) indicates start-up element from element (900) once control module (50) has been powered on. Block (1102) indicates the step where the operator attaches control cord receptacle (57) and control cord (28) as described above with respect to element (910). Block (1104) indicates that the operator attach probe (12) to holster (14) as described above with respect to element (92) 0. Block (1106) indicates that the operator to attach vacuum assembly (70) to vacuum receptacle (102) as described above with respect to element (930). Block (1108) indicates that the operator attaches vacuum assembly (70) to vacuum canister (62) as described above with respect to element (940). Once vacuum assembly (70) is attached to vacuum canister (62), block (1110) indicates that the initialization process can begin as described above with respect to element (960). Once initialization is complete, visualization (54) may automatically transition to sampling element (970) as indicated with block (1112).

### Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

An apparatus, the apparatus comprising: a biopsy device; a valve assembly configured to be in fluid communication with the biopsy device, and a control module configured to be in electrical communication with the biopsy device and in fluid communication with the valve assembly, the control module including a display configured to provide instructions for: placing the biopsy device in electrical communication with the control module, inserting the valve assembly into the control module, and placing the valve assembly in fluid communication with the control module.

### EXAMPLE 2

The apparatus of example 1, the biopsy device comprising a control unit configured to be in electrical communication with the control module.

### EXAMPLE 3

The apparatus of example 2, the control module being configured to detect when in electrical communication with the control unit, the display being configured to communicate to an operator whether the control unit is in electrical communication to the control module.

### EXAMPLE 4

The apparatus of example 3, the control module being configured to make the biopsy device inoperable if electrical communication with the control unit is not detected.

### EXAMPLE 5

The apparatus of example 2, the biopsy device further comprising a probe in fluid communication with the valve assembly, the control unit being removably coupled to the probe.

### EXAMPLE 6

The apparatus of example 5, the control module being configured to detect whether the probe has been removably coupled to the control unit, the display being configured to communicate to an operator whether the probe has been removably coupled to the control unit.

### EXAMPLE 7

The apparatus of example 5, the control module being configured to make the biopsy device inoperable when the probe is uncoupled from the control unit.

### EXAMPLE 8

The apparatus of example 1, the control module being configured to detect when the control module is in fluid communication with the valve assembly.

### EXAMPLE 9

The apparatus of example 8, the display being configured to communicate to an operator whether the control module is in fluid communication with the valve assembly.

### EXAMPLE 10

The apparatus of example 1, the valve assembly being configured to be removably coupled to the control module.

### EXAMPLE 11

The apparatus of example 10, the control module being configured to detect whether the valve assembly is coupled to the control module.

### EXAMPLE 12

The apparatus of example 11, the display being configured to communicate to an operator whether the valve assembly is coupled to the control module.

### EXAMPLE 13

The apparatus of example 11, the control module comprising a switch configured to contact the valve assembly to thereby detect whether the valve assembly is coupled to the control module.

### EXAMPLE 14

The apparatus of example 1, the biopsy device comprising a control unit and a probe, the control unit being removably coupled to the probe, the display being further configured to provide instructions for coupling the control unit to the probe.

### EXAMPLE 15

The apparatus of example 1, the display being configured to provide confirmation that: the biopsy device is in electrical communication with the control module, the valve assembly has been inserted into the control module, and the valve assembly is in fluid communication with the control module.

### EXAMPLE 16

A method, the method comprising: sensing that a valve assembly of a biopsy device is not coupled with a valve receptacle of a control module, displaying instructions to couple the valve assembly to the valve receptacle when it is sensed that the valved assembly is not coupled with the valve receptacle; sensing that the valve assembly is coupled with the valve receptacle, enabling a valve of the valve assembly by using the valve receptacle once the valve assembly is sensed being coupled with the valve receptacle, the valve being in fluid communication with the biopsy device.

### EXAMPLE 17

The method of example 16, the valve assembly being configured to be in fluid communication with a vacuum assembly of the control module, the method further comprising detecting that the valve assembly is in fluid communication with the vacuum assembly.

### EXAMPLE 18

The method of example 17, the method further comprising disabling the biopsy device once it is detected that the valve assembly is no longer in fluid communication with the vacuum assembly.

### EXAMPLE 19

The method of example 16, the valve receptacle comprising a switch, the method further comprising engaging the switch with the valve assembly to sense that the valve assembly is coupled with the valve receptacle of the control module.

### EXAMPLE 20

An apparatus, the apparatus comprising: a biopsy device; a valve assembly configured to be in fluid communication with the biopsy device and including a valve, the valve being configured to provide vacuum to the biopsy device, a control module comprising a valve receptacle and a power coupling, the valve receptacle comprising a valve coupling, the power coupling being configured to couple with the biopsy device to make the biopsy device operable, the valve receptacle being configured to couple with the valve assembly, the valve receptacle comprising a sensor configured to detect the coupling of the valve assembly to the valve receptacle, the valve coupling being configured to permit operation of the valve to provide vacuum to the biopsy device once the valve assembly has been detected by the sensor.

### EXAMPLE 21

An apparatus, the apparatus comprising: a biopsy device; a valve assembly configured to be in fluid communication with the biopsy device, and a control module configured to be in electrical communication with the biopsy device and the control module being further configured to be in fluid communication with the valve assembly, the control module including a display capable of providing instructions to an operator.

### EXAMPLE 22

The apparatus of example 21, the display being configured to provide a first set of instructions for placing the biopsy device in electrical communication with the control module.

### EXAMPLE 23

The apparatus of example 21, the display being configured to provide a first set of instructions for inserting the valve assembly into the control module.

### EXAMPLE 24

The apparatus of example 21, the display being configured to provide a first set of instructions for placing the valve assembly in fluid communication with the control module.

### EXAMPLE 25

The apparatus of example 21, the biopsy device comprising a holster and a probe, the probe being configured to be coupled to the holster, the display being configured to provide a first set of instructions for coupling the probe to the holster.

### EXAMPLE 26

The apparatus of any of examples 22 through 25, in which a second set of instructions are provided on the display.

### EXAMPLE 27

The apparatus of any of examples 22 through 25, in which a second set of instructions are provided on the display, the second set of instructions being instructions for: placing the biopsy device in electrical communication with the control module, inserting the valve assembly into the control module, placing the valve assembly in fluid communication with the control module, or coupling the probe to the holster.

### EXAMPLE 28

The apparatus of example 21, the display being configured to provide four sets of instructions simultaneously, the sets of instructions being: placing the biopsy device in electrical communication with the control module, inserting the valve assembly into the control module, placing the valve assembly in fluid communication with the control module, or coupling the probe to the holster.

### Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. An apparatus, the apparatus comprising:
(a) a biopsy device;
(b) a valve assembly configured to be in fluid communication with the biopsy device, and
(c) a control module configured to be in electrical communication with the biopsy device and in fluid communication with the valve assembly, the control module including a display configured to provide instructions for:
(i) placing the biopsy device in electrical communication with the control module,
(ii) inserting the valve assembly into the control module, and
(iii) placing the valve assembly in fluid communication with the control module.

2. The apparatus of claim 1, the biopsy device comprising a control unit configured to be in electrical communication with the control module.

3. The apparatus of claim 2, the control module being configured to detect when in electrical communication with the control unit, the display being configured to communicate to an operator whether the control unit is in electrical communication to the control module.

4. The apparatus of claim 3, the control module being configured to make the biopsy device inoperable if electrical communication with the control unit is not detected.

5. The apparatus of claim 2, the biopsy device further comprising a probe in fluid communication with the valve assembly, the control unit being removably coupled to the probe.

6. The apparatus of claim 5, the control module being configured to detect whether the probe has been removably coupled to the control unit, the display being configured to communicate to an operator whether the probe has been removably coupled to the control unit.

7. The apparatus of claim 5, the control module being configured to make the biopsy device inoperable when the probe is uncoupled from the control unit.

8. The apparatus of claim 1, the control module being configured to detect when the control module is in fluid communication with the valve assembly.

9. The apparatus of claim 8, the display being configured to communicate to an operator whether the control module is in fluid communication with the valve assembly.

10. The apparatus of claim 1, the valve assembly being configured to be removably coupled to the control module.

11. The apparatus of claim 10, the control module being configured to detect whether the valve assembly is coupled to the control module.

12. The apparatus of claim 11, the display being configured to communicate to an operator whether the valve assembly is coupled to the control module.

13. The apparatus of claim 11, the control module comprising a switch configured to contact the valve assembly to thereby detect whether the valve assembly is coupled to the control module.

14. The apparatus of claim 1, the biopsy device comprising a control unit and a probe, the control unit being removably coupled to the probe, the display being further configured to provide instructions for coupling the control unit to the probe.

15. The apparatus of claim 1, the display being configured to provide confirmation that:
(a) the biopsy device is in electrical communication with the control module,
(b) the valve assembly has been inserted into the control module, and
(c) the valve assembly is in fluid communication with the control module.

16. A method, the method comprising:
(a) sensing that a valve assembly of a biopsy device Is not coupled with a valve receptacle of a control module,
(b) displaying instructions to couple the valve assembly to the valve receptacle when it is sensed that the valved assembly is not coupled with the valve receptacle;
(c) sensing that the valve assembly is coupled with the valve receptacle,
(d) enabling a valve of the valve assembly by using the valve receptacle once the valve assembly is sensed being coupled with the valve receptacle, the valve being m fluid communication with the biopsy device.

17. The method of claim 16, the valve assembly being configured to be in fluid communication with a vacuum assembly of the control module, the method further comprising detecting that the valve assembly is in fluid communication with the vacuum assembly.

18. The method of claim 17, the method further comprising disabling the biopsy device once it is detected that the valve assembly is no longer in fluid communication with the vacuum assembly.

19. The method of claim 16, the valve receptacle comprising a switch, the method further comprising engaging the switch with the valve assembly to sense that the valve assembly is coupled with the valve receptacle of the control module.

20. An apparatus, the apparatus comprising:
(a) a biopsy device;
(b) a valve assembly configured to be in fluid communication with the biopsy device and including a valve, the valve being configured to provide vacuum to the biopsy device,
(c) a control module comprising a valve receptacle and a power coupling, the valve receptacle comprising a valve coupling, the power coupling being configured to couple with the biopsy device to make the biopsy device operable, the valve receptacle being configured to couple with the valve assembly, the valve receptacle comprising a sensor configured to detect the coupling of the valve assembly to the valve receptacle, the valve coupling being configured to permit operation of the valve to provide vacuum to the biopsy device once the valve assembly has been detected by the sensor.

21-28. (canceled)
